(19) United States
(12) Patent Application Publication
(10) Pub. No.: US 2004/0030124 A1
(43) Pub. Date: Feb. 12, 2004

(54) Process for recovering chlorosucrose compounds

(76) Inventors: Steven J. Catani, Athens, GA (US); Mark R. Huber, Mobile, AL (US); Nicholas M. Vernon, Daphne, AL (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/213,387

(22) Filed: Aug. 6, 2002

(51) Int. Cl. C07H 3/00

(52) U.S. Cl. 536/123.13

(57) ABSTRACT

This invention relates to a crystallization process for sucralose in which a mother liquor is obtained by separating crystalline sucralose from the remaining liquid. A portion of this mother liquor is returned to the same crystallization stage; the amount returned is adjusted so as to maintain the overall related substances level in the process below levels at which yield declines significantly.

### FIELD OF THE INVENTION

This invention relates to a process for recovering sucralose from related substances by crystallization. A solution containing sucralose and related substances is introduced into a suitable vessel and subjected to conditions that cause crystallization of the desirable compound. A portion of the crystal slurry thus formed is removed and separated into a solid crystal phase and a residual mother liquor. A portion of the mother liquor is returned to the original crystallizer vessel. The amount of mother liquor returned is controlled so as keep the overall related substances level during the crystallization below a threshold level that permits a high yield in the crystallization process.

### BACKGROUND OF THE INVENTION

Sucralose, 4,1′,6′-trichloro-4,1′,6′-trideoxygalactosucrose, a sweetener with a sweetness intensity several hundred times that of sucrose, is made from sucrose by replacing the hydroxyl groups in the 4, 1′, and 6′ positions with chlorine. Synthesis of sucralose is technically challenging because of the need to selectively replace specific hydroxyl groups with chlorine atoms, while preserving other hydroxyl groups including a highly reactive primary hydroxyl group. Numerous approaches to this synthesis have been developed. See, e.g. U.S. Pat. Nos. 4,362,869, 4,826,962, 4,980,463, and 5,141,860, which are expressly incorporated herein by reference. However, such approaches typically yield a product that contains related substances in addition to sucralose.

Producing sucralose having low levels of related substances involves a number of sequential recovery steps. In brief, an initial non-crystallization approach, e.g., solvent to solvent extraction, may be used to separate sucralose from other compounds that differ in their polarity. The sucralose obtained from this step may then be subjected to a number of sequential crystallization steps.

Crystallization is widely used to recover compounds, including, but not limited to, sugar, sucralose, and related substances. Crystallization is carried out by inducing the formation of crystals in a solution, followed by separating the crystals from the remaining solution (the “mother liquor”), i.e., recovering.

In processes for making highly valuable compounds, such as sucralose, the efficient recovery of such desired compounds is important. Therefore, it is usually desirable to recover the largest possible quantity of such desired compounds, e.g., sucralose, at each crystallization step. However, those skilled in the art will readily recognize that crystallization entails a loss of valuable end product, since the yield will generally be less than 100%. Specifically with regard to sucralose, while not being bound to theory, it is believed that this may be caused by the high solubility of sucralose in water, which is a typical solvent that can be used to crystallize sucralose. Such solubility limits the single pass yield. Other solvents, e.g., ethyl acetate, have better solubility curves, but are less desirable to crystallize sucralose due to lower crystallization selection and the off-tastes in the resultant product.

In a sequential crystallization, the sucralose crystal material obtained at each crystallization step is dissolved in water and an additional crystallization is performed on the solution thereby obtained. After each subsequent crystallization, the sucralose crystals obtained will contain lower amounts of related substances, since there are less related substances present that could be potentially introduced into the crystals. Similarly, the mother liquor at each stage also has a lower ratio of related substances to sucralose than the mother liquors obtained at earlier crystallization steps.

Because a considerable capital investment is involved in the equipment for carrying out these sequential purification steps, those skilled in the art will also recognize that processes that increase yield and improve throughput of equipment will have a strong commercial value.

Two routes are known for recovering additional product from the mother liquor. One route is to subject the mother liquor to conditions such as solvent removal or decreased temperature that will promote the formation of additional crystals. A second route is to recycle the mother liquor to an earlier crystallization stage. The use of these procedures in the purification of sucralose is disclosed in pending U.S. application Serial No. 10/092,730. The general use of these procedures in crystallization purification are disclosed in pp. 243-285 of the Kirk-Othmer Encyclopedia of Chemical Technology, vol. 7, John Wiley & Sons, which is incorporated herein by reference.

The first method described above to meet those commercial considerations is to induce the formation of additional crystals from the mother liquor (this is known in the art as “recropping”). Generally, the crystals obtained from crystallizing the mother liquor will have more related substances than those obtained in crystallizing the virgin solution, since the related substance level in the mother liquor is greater than that of the virgin solution. Additionally, the yield (expressed as amount of desirable compound, e.g., sugar, sucralose, related compounds, etc., obtained by crystallization, divided by the total amount of desirable compound present) is lower in recropping because the increased related substances level interferes with the extent of crystallization.

The latter method described above is disclosed in presently pending U.S. application Serial No. 10/092,730, wherein the mother liquor may be advantageously returned to an earlier crystallization or to a liquid to liquid extraction stage, since the mother liquor may contain lower amounts of related substances than the in-process materials at the earlier stage.

An additional method not indicated or suggested in the above disclosures is to return a portion of the mother liquor to the same crystallization step while maintaining commercially acceptable yield (“recycled”). This return of the mother liquor to the same crystallization process step from which it originated is one aspect of the current invention. Such a method would appear to be technically undesirable because it have the effect of increasing the related substances level present at that stage, with a consequent decrease in yield of sucralose crystals.

Surprisingly, however, the inventors have found that a significant portion of the mother liquor can be recycled to increase yield and reduce related substances level in the end product, e.g., sucralose. In fact, it has been discovered that, up to a certain level, the impact of related substances on yield is insignificant. The mother liquor recycling to the same crystallization step is made possible by the discovery of the non-linear nature of the yield to related substance curve. Therefore, it is possible and desirable to recycle a portion of the mother liquor back into the crystallization step from which it originated. This mode of recycling the mother liquor reduces the capital investment needed to produce sucralose on a commercial scale.

Such mother liquor recycling reduces the capital investment needed to produce commercially pure sucralose and can be integrated into a crystallization process, in which virgin solution and returned mother liquor are introduced into a process in which crystallization is carried out.

The key parameter in successfully carrying out the processes of this invention has been identified as controlling the level of related substances in the crystallization vessel and in the total input feed. It has been found that the overall related substances level in the crystallization vessel must be controlled to be at or below a threshold level at which the crystal yields begin to decrease substantially. It has also been surprisingly found that, although related substances level in the crystallizer may be monitored, such monitoring is not necessary and is in fact difficult to perform due to the unsteady state of the crystal slurry and two phase nature for the crystal slurry. Rather, the related substances level of the virgin solution is monitored and has been related directly to an optimum sucralose yield through an empirical equation. Such relationship has been found to not be linear and, above a threshold related substances level, the crystal yield declines markedly.

### SUMMARY OF THE INVENTION

One embodiment of the present invention is directed to a method for recovering sucralose from a solution having at least sucralose and related substances, the process including the following sequential steps: providing a virgin solution comprising at least sucralose and related substances; inducing the formation of sucralose crystals from the virgin solution to yield a slurry; removing a portion of the slurry; separating crystalline sucralose and a mother liquor from the portion of the slurry; recycling a portion of the mother liquor into the remaining slurry along with an additional portion of virgin solution to produce an admixture solution, wherein the portion of the recycled mother liquor is a positive number less than or equal to 0.9933−0.0321x−0.0126x², wherein x is a number between 0 and 100 and is obtained by taking a value provided by dividing the weight percent of related substances in the virgin solution by the sum of the weight percent of sucralose and related substances in the virgin solution and multiplying the value by 100; and repeating steps (b)-(d) at least once.

Another embodiment of the present invention is directed to a method for improving the yield of crystalline sucralose in a process for recovering crystalline sucralose from a solution having at least sucralose and related substances, the process including the following sequential steps: providing a virgin solution comprising at least sucralose and related substances; inducing the formation of sucralose crystals from the virgin solution to yield a slurry; removing a portion of the slurry; separating crystalline sucralose and a mother liquor from the portion of the slurry; recycling a portion of the mother liquor into the remaining slurry along with an additional portion of virgin solution to produce an admixture solution, wherein the total related substances level relative to sucralose plus related substances in the slurry is maintained from about 6% to about 9% on a weight basis; and repeating steps (b)-(d) at least once.

An additional embodiment of the present invention is directed to a method for enhancing the yield of crystalline sucralose from an admixture solution having at least sucralose, related substances, and a recycled mother liquor in a continuous process operation including the following sequential steps: determining the related substances level in an admixture solution comprising at least sucralose, related substances, and recycled mother liquor; providing the admixture solution into a continuous process crystallizer vessel; inducing the formation of sucralose crystals in the admixture solution to yield a slurry comprising at least sucralose crystals, sucralose, and related substances; removing a portion of the slurry; separating crystalline sucralose and a mother liquor from the portion of the slurry; determining the related substances level of the mother liquor; and recycling a portion of the mother liquor to the continuous process crystallizer vessel such that the total level of related substances level in the crystallizer vessel relative to sucralose plus related substances is about 6% to about 9% on a weight basis.

Yet another embodiment of the present invention is directed to a method for enhancing the yield of crystalline sucralose from an admixture solution having at least sucralose, related substances, and a recycled mother liquor in a continuous process operation including the following sequential steps: determining the related substances level in an admixture solution comprising at least sucralose, related substances, and recycled mother liquor; providing the admixture solution into a continuous process crystallizer vessel; inducing the formation of sucralose crystals in the admixture solution to yield a slurry comprising at least sucralose crystals, sucralose, and related substances; removing a portion of the slurry; separating crystalline sucralose and a mother liquor from the portion of the slurry; determining the related substances level of the mother liquor; and recycling a portion of the mother liquor to the continuous process crystallizer vessel such that the portion of the recycled mother liquor is a positive number less than or equal to 0.9933−0.0321x−0.0126x , wherein x is a number between 0 and 100 and is obtained by taking a value provided by dividing the weight percent of related substances in the virgin solution by the sum of the weight percent of sucralose and related substances in the virgin solution and multiplying the value by 100.

Another embodiment of the present invention is directed to a composition having a solution free of solids including at least sucralose and related substances, wherein the level of related substances is above the level that suppresses crystal formation.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### BRIEF DESCRIPTION OF THE DRAWINGS

FIG. 1 is a schematic presentation of one embodiment of the methods of the present invention for recovering sucralose from a feeds having sucralose and related compounds wherein the mother liquor is recycled to the same crystallization step.

FIG. 2 provides a chart that depicts the relationship between the chlorosucrose related substances level and the yield of crystalline sucralose in a crystallization process.

FIG. 3 provides a chart that depicts the optimum mother liquor recycle level as a function of the feed related substances level.

FIG. 4 depicts the relationship between feed chlorosucrose related substances levels, the portion of the mother liquor recycled, and the overall system yield obtained at these related substances levels and recycle amounts.

FIG. 5 is a graphic presentation of the empirical mathematical relationship between the related substances levels of the fresh feed and the portion of mother liquor recycle that provides optimal yield.

### DETAILED DESCRIPTION OF THE INVENTION

It is understood that the present invention is not limited to the particular methodologies, protocols, solvents, and reagents, described herein, as these may vary. It is also to be understood that the terminology used herein is used for the purpose of describing particular embodiments only, and is not intended to limit the scope of the present invention. It is noted that as used herein and in the appended claims, the singular forms “a,” “an,” and “the” include plural reference unless the context clearly dictates otherwise. Thus, for example, a reference to “a solvent” is a reference to one or more solvents and includes equivalents thereof known to those skilled in the art and so forth.

Unless defined otherwise, all technical and scientific terms used herein have the same meanings as commonly understood by one of ordinary skill in the art to which this invention belongs. Preferred methods, devices, and materials are described, although any methods and materials similar or equivalent to those described herein can be used in the practice or testing of the present invention. All references herein cited are incorporated by reference herein in their entirety.

Definitions

Batch process: as used herein, describes a procedure in which a fixed amount of materials are introduced into a process, and the products obtained from this fixed amount of input are recovered prior to the addition of more input material.

Continuous process: as used herein, includes procedures in which product may be removed from the process while input may be added; removal of product or addition of input may be incremental, discontinuous, or at a constant rate. Those skilled in the art will readily recognize that the terms “batch operation” and “continuous operation” are somewhat arbitrary, and that many intermediate operations between pure batch operations and pure continuous processes are possible. The embodiments of the present invention may be readily practiced by this full range of possible operations.

Crystallization: as used herein includes processes in which a solution is rendered saturated or supersaturated with respect to a dissolved component, and the formation of crystals of this component is achieved. The initiation of crystal formation may be spontaneous, or it may require the addition of seed crystals. As used herein, crystallization also describes the situation in which a solid or liquid material is dissolved in a solvent to yield a solution that is then rendered saturated or supersaturated so as to obtain crystals. Also, included in the term crystallization are the ancillary processes of washing the crystals with one or more solvents, drying the crystals, and harvesting the final product so obtained.

Mother liquor: as used herein, includes the solution remaining after a crystallization step has been performed. Mother liquor may be obtained by separating a crystal slurry into a solid phase and a liquid phase. The remaining liquid phase, which still contains desirable product, is the mother liquor.

Related substance(s): as used herein, broadly describes compounds related to sucralose, and includes both compounds in which the original structural conformation of sucrose is retained, as well as compounds in which the sucrose molecule has undergone changes, such as the formation of galactosucrose or anhydro- related compounds. Examples of related substances include 4,6,6′-trichlorosucrose, 4,1′,6′-trichlorosucrose, 6,1′,6′-trichlorosucrose, 4,6′-dichlorogalactosucrose, 1′,6′-dichlorosucrose, and 3′,6′-dichloroanhydrosucrose but many additional related substances are known or may exist. This term also includes sucralose and other chlorinated sugars on which some or all of the hydroxyl groups remaining after chlorination may have been blocked by esterification or other means known to those skilled in the art. Also may be called chlorosucrose related substances.

Recycled mother liquor: as used herein refers to the practice of adding the mother liquor to another sucralose-containing solution prior to, or during, its crystallization. The mother liquor may be further concentrated or purified prior to recycling.

Semi-continuous process: as used herein describes a procedure in which a fixed amount of materials are introduced into a process, and the products obtained from this fixed amount of input are recovered prior to the addition of more input material. Those skilled in the art will readily recognize that the terms “semi-continuous process” and “continuous process” are somewhat arbitrary, and that many intermediate processes between pure semi-continuous processes and pure continuous processes are possible. The embodiments of the present invention may be readily practiced by this full range of possible processes.

Yield: as used herein, refers to the amount of crystalline sucralose obtained from an operation, divided by the input amount of sucralose, on a weight to weight basis.

Sucralose can be synthesized from sucrose by the procedure disclosed in U.S. Pat. Nos. 5,470,969, 5,498,709, 4,977,294, 4,980,463, and 5,136,031, which are incorporated herein by reference. However, any method to synthesize sucralose can also be used, such as those described in U.S. Pat. Nos. 4,950,746, 5,023,329, 5,298,611, and 5,530,106, which are incorporated herein by reference.

The solution resulting from the above procedures can be subjected to solvent extraction procedures, as described in pending U.S. application Serial No. 10/092,715, which is incorporated herein. The resulting extracted solution contains, among other things, sucralose and related substances as a solution in ethyl acetate. Sucralose crystallized from the extracted solution is disclosed in pending U.S. application Serial No. 10/092,730, which is incorporated herein.

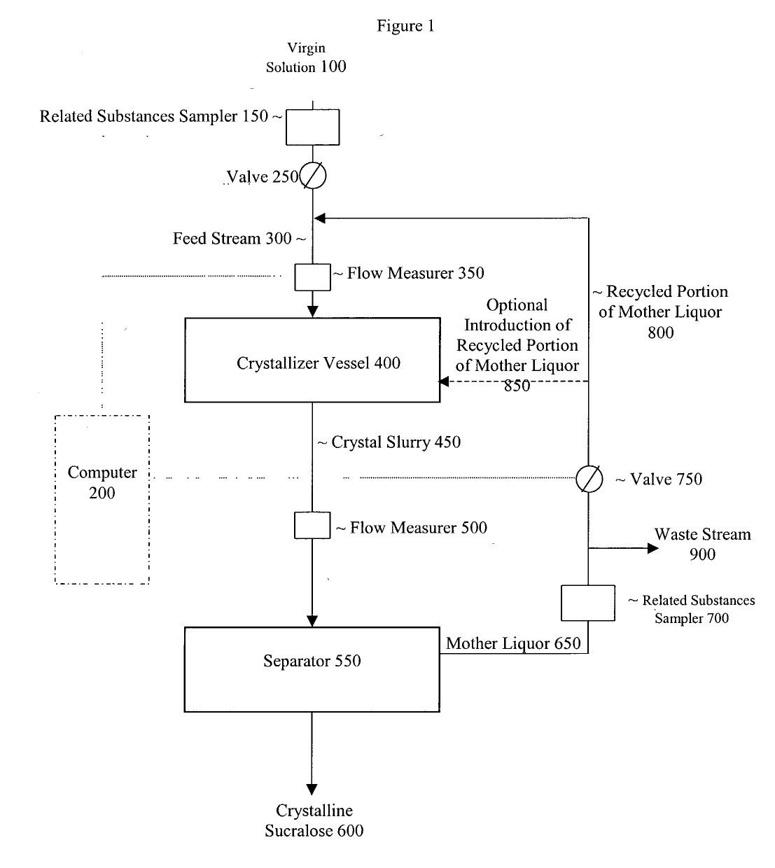

FIG. 1 depicts one embodiment of the invention where the mother liquor is recycled to the same crystallization step and crystalline sucralose may be recovered by crystallization. In this embodiment, the sucralose obtained by taking the sucralose stream from extraction step, and crystallizing it once by evaporative concentration of an ethyl acetate solution, containing, among other things, sucralose, related substances, residual solvent (primarily ethyl acetate) and some inorganic salts, is dissolved in water to form virgin solution 100, which is used as feed for the next stage. The primary related substance is usually 4,6,6′-trichlorosucrose and two other related substances, i.e., 6,1′,6′-trichlorosucrose and 4,6′-dichlorogalactosucrose, are also typically present.

A portion of virgin solution 100, e.g., 100 ml, may be sampled manually or automatically and measured by a sampler 150. Such sampling may be conducted periodically, e.g., every hour, every two hours, every six hours, and every twelve hours, to measure concentration. The samples thus taken may be analyzed by any technique that is able to measure and identify compounds in solution, e.g., by high performance liquid chromatography (“HPLC”). If HPLC is used, a Shodex laser light scattering detector may be used. Specifically, one or more related substances may be measured as the percent of each related substance relative to all the chlorosucroses, including sucralose, present in virgin solution 100. Optionally, the resulting data may be sent to computer 200 and stored for future use.

The virgin solution 100 is then released by valve 250, to form a feed stream 300. The flow rate of feed stream 300 may be measured by flow measurer 350, and the data regarding this flow rate may optionally be stored in computer 200 for later use. The feed stream 300 may then be introduced into a crystallizer vessel 400 where crystallization of sucralose may be induced by known crystallization methods, including, but not limited to evaporative removal of water, cooling, membrane concentration and the like. A crystal slurry, having, among other things, crystalline sucralose and a mother liquor, may be formed in the crystallizer vessel 400 and may be kept in a suspended state by continuous pumping and agitation.

The data regarding the flow rate of feed stream 300 may be used to allow the amount of crystal slurry 450 removed to be equal or proportional to the amount of feed stream 300 entering the crystallizer vessel 400.

Virgin solution 100 from the upstream crystallization operation is continuously or intermittently introduced into the crystallizer vessel 400, and periodically determined portions of crystal slurry 450 are removed from the crystallizer vessel 400. This determination of steady state may be remote level indication and local visual assessment and controlled by, for example, feed 300 or slurry 450 remove rate flow adjustments. A generally steady state condition was achieved, wherein the amount of virgin solution 100 in the feed 300 was approximately equal to the amount of crystal slurry 450 removed from the crystallizer vessel 400. Such adjustments may be made manually or by using automatic flow controls.

The portion of crystal slurry 450 removed may then be separated 550 into crystalline sucralose 600, and mother liquor 650, which is substantially free of sucralose crystals. Mother liquor 650 may be sampled and measured by sampler 700 for its related substances level. The appropriate amount of mother liquor 650 to recycle is calculated from the related substances level of the virgin solution 100. The remainder of the mother liquor 650 is sent to waste stream 900, or is returned to a pre-crystallization purification process, e.g., solvent to solvent extraction, or an earlier crystallization stage. An example of a suitable pre-crystallization purification process is provided in pending U.S. application Serial No. 10/092,730, filed Mar. 8, 2002, which is expressly incorporated by reference herein.

Specifically, one or more related substances may be measured as the percent of each related substance relative to all the chlorosucroses, including sucralose, present in the mother liquor 650. The data obtained may, optionally, be sent to computer 200, and stored for future use. A determined portion of the mother liquor may be released by valve 750 and recycled 800 to the feed stream 300.

Specifically, the related substances level is determined in the crystallizer vessel 400, the feed stream 300, or both.

The amount of recycled mother liquor 800 may be equal to a positive number less than or equal to 0.956−0.0148x2−0.0125x, where x is a number between 0 and 100 and represents the percentage of total related substances present in virgin solution 100.

Optionally or concurrently, a portion of the recycled mother liquor 850 may be added directly to the crystallizer vessel 400 as illustrated by the dashed arrow in FIG. 1. The portion of mother liquor not recycled 800 to the feed stream 300 or introduced 850 to the crystallizer 400 can be discarded in the waste stream 900 or subjected to other purification procedures. Crystalline sucralose 600 may be further purified or used in other applications, depending on its related substances level and the desired end use.

Additional virgin solution 100 and a portion of the mother liquor 800, 850 is returned to the crystallization vessel resulting in an admixture solution in the crystallizer vessel 400. In other embodiments not represented by FIG. 1, additional virgin solution 100 and a portion of the mother liquor 800, 850 may be introduced separately in the crystallizer vessel 100.

In other embodiments, the process of the present invention may be carried out in a batch or in a semi-continuous process.

One parameter in the processes of this invention is monitoring the overall level of related substances in the virgin solution or the total crystallizer content, i.e., the total feed in a batch process before any crystals are formed or the total contents of the slurry, which include the mother liquor in the crystallizer after the mother liquor is recycled and the virgin solution, in a continuous process. This monitoring may be performed by taking samples from each stage of the crystallization process.

The overall related substances level in the crystallizer vessel 400 is maintained at or below a threshold related substances level at which the crystal yield decreases substantially. The relationship between crystal yield and related substances level of the solution is not linear. The yield of crystalline product markedly decreases if the feed stream is above the determined threshold related substances level. An empirical equation was derived to determine the portion of mother liquor that can be recycled to the crystallizer. The portion is dependent on the related substances level of the feed stream and the desired percent yield of crystalline sucralose from the crystallizer. Also, the curve derived from the equation is used to predict the recycle level after which any improvement in yield diminishes.

Achieving such a steady state in the crystallizer permitted experimentation to determine the effects of related substances levels on the yield of sucralose crystals. The related substances level was controlled by monitoring the related substances level of virgin solution entering the system, and blending into this, mother liquor from the crystallization stage. Optionally, the related substances level could be monitored in the total solution volume of the crystallizer vessel 400, i.e., virgin solution 100, mother liquor 650, and remaining crystal slurry 450.

The yield was expressed as the percent by weight of crystalline sucralose of the total amount sucralose present in the slurry removed, i.e., the total amount of sucralose present was the amount in the crystal state plus the amount remaining in solution. The yield was evaluated as a function of the total related substances level in the crystallizer vessel 400, which is identical to the related substances level of the contemporaneously removed crystal slurry 450, since continuous agitation resulted in content uniformity.

Data were collected during an extended production campaign and the average weekly yield data was analyzed as a function of the average related substances level during that week. The weekly data so obtained are presented in the table below. The data comparing very low related substances levels, e.g., about 0%, were obtained by adding a solution of previously purified sucralose to the crystallizer vessel 400.

TABLE

| Related substances | | |
|---|---|---|
| Level in Mother Liquor | Total Related substances | |
| (%) | in Crystallizer Feed (%) | Yield (%) |
| 0 | 0 | 65 |
| 7.2 | 2.66 | 63 |
| 8.4 | 3.19 | 62 |
| 8.6 | 3.10 | 64 |
| 8.6 | 2.92 | 66 |
| 9.1 | 3.28 | 64 |
| 12.2 | 5.25 | 57 |
| 13.5 | 5.80 | 57 |
| 14 | 6.72 | 52 |

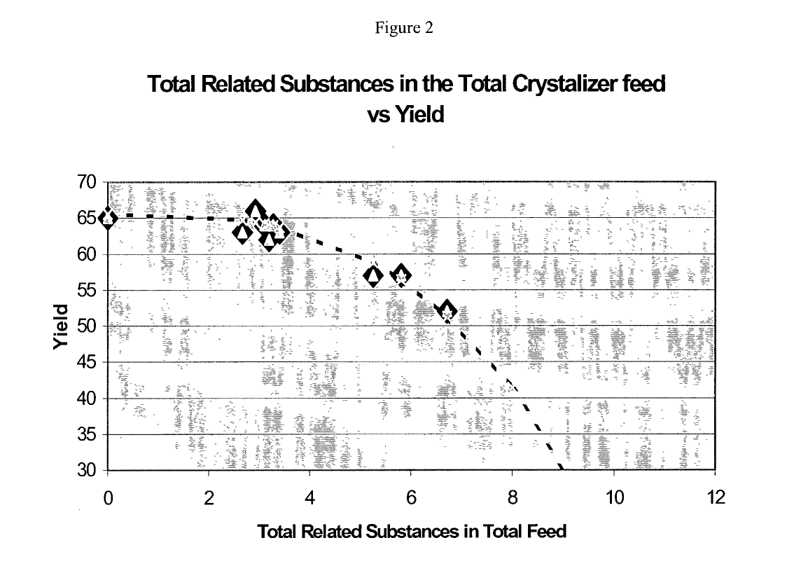

FIG. 2 presents graphically the observed relationship between percent yield and the related substances level. The observed relationship was non-linear, with the related substances level in the total crystallizer feed of up to about 3% having relatively little effect on yield, which remained in the range of about 62% to about 66%. In contrast, total related substances levels exceeding about 3% resulted in a marked reduction in yield. This data was fit to an equation using Microsoft® Excel 2000 software and yield the following equation

Yield=65.6−0.07246·x³

Where x is a number between 0 and 100 representing the percent total related substances relative to all the chlorinated carbohydrates, i.e., sucralose and related substances, found in the total crystallizer vessel contents. Based on this formula and its underlying data, Microsoft® Excel 2000 software was used to derive an equation that determines the maximum proportion (as a percent value) of mother liquor that can be returned to the crystallizer vessel while maintaining a specific percent yield. The equation thus obtained was:

Maximum recycle portion=0.9933−0.0321x−0.0126x²

Where x is a number between 0 and 100 representing the percent total related substances relative to all the chlorinated carbohydrates, i.e., sucralose and related substances, found in the feed stream.

Some values of x will obviously generate negative values for the maximum recycle portion, which would indicate related substances levels at which no crystal slurry would form. While not relevant to maximizing yield, this aspect of the invention is useful where a stable solution of sucralose that does not form a slurry is desired.

For the purpose of mother liquor recycle calculations, negative numbers are meaningless and therefore, the maximum recycle portion is equal to the greater of zero and the value obtained by solving the above equation for x. The maximum recycle portion obtained from this equation represents the maximum portion of the mother liquor that can be returned to the same stage of crystallization without suffering a commercially undesirable loss of yield.

The level of related substances present in the mother liquor and in the admixture solution can be measured and maintained using, for example, taking samples and using flow rate measurers. By using these methods, the related substances level in the crystallizer can be maintained at the threshold level where the recovery of crystalline sucralose is maximized.

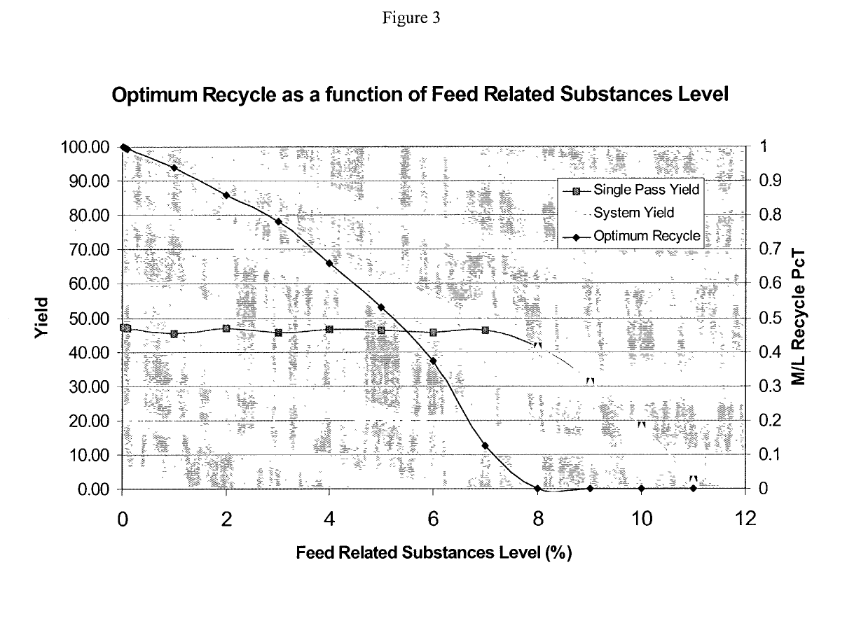

FIG. 3 depicts the interactions between feed related substances levels and the single pass yield, the system yield, and the optimal portion of mother liquor for recycling. The feed in this figure is a mixture of virgin solution and mother liquor from the crystallization stage into which the feed is being introduced. The single pass yield is determined by dividing the amount of crystalline sucralose obtained by the amount of sucralose present in the feed to this step. Determination of the system yield requires measuring the amount of sucralose present in the portion of the mother liquor that is not returned to this crystallization step. This amount is then subtracted from the total amount of sucralose introduced by the feed stream. The number so obtained is then divided by the total amount of sucralose introduced by the feed to provide the system yield. The optimum recycle is the portion of the mother liquor that can be recycled to achieve the single pass yield and system yield as shown in the figure.

The results depicted in FIG. 3 show that as the feed stream related substances level increases above about 7%, the single pass yield declines markedly, and the rate of decrease in system yield as a function of related substances level also increases. This indicated that the amount of mother liquor returned must be adjusted to maintain the feed related substances level below about 8%, with optimal performance being obtained at somewhat lower related substances levels, e.g., about 7-7.5%.

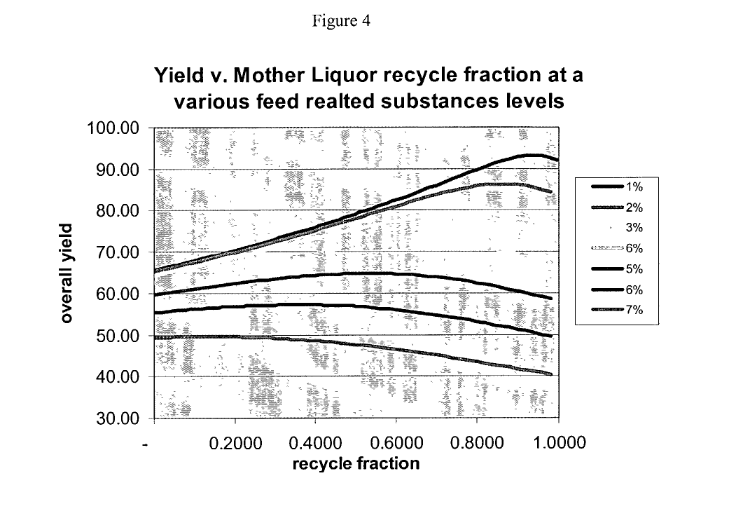

FIG. 4 presents a graphical depiction that was used in a manufacturing operation to optimize overall process yield. The related substances level of the virgin solution was determined, and based on this the portion of mother liquor that must be recycled to obtain a specific yield was interpolated from the graph. Of course, the manufacturing operation would be configured to achieve maximal yield, and therefore the recycle level that provided the highest yield would generally be accepted.

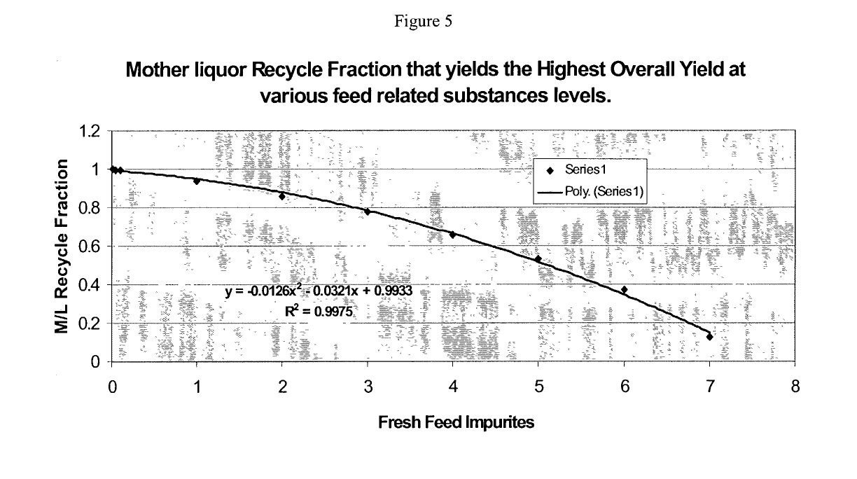

FIG. 5 depicts a graphical presentation of the empirical equation discussed above. This depicts, for any given related substances level in the virgin solution, the portion of mother liquor that could be recycled to achieve the highest overall system yield of crystalline sucralose. The equation represents the amount of recycled mother liquor that can be added to the virgin solution to maintain the threshold related substances level in the crystallizer that may maximize yield of crystalline sucralose. Additional process alternatives that can achieve the same result include, among other things, adding the recycled mother liquor to the crystallizer separately from the virgin solution entering the crystallizer or combining a portion of the mother liquor to the virgin solution and adding another portion of the mother liquor.

The above results were based on the measurement on the total levels of related substances present in the various streams. Similar analyses were conducted with specific related substances. Notably, levels of the predominant related substance, 4,6,6′-trichlorosucrose, showed a similar non-linear relationship between increasing levels of this specific compound and decreased yield. As would be expected, minor related substances had minimal impact on the yield. However, analysis using the total related substances content provided the most consistent data for the amount of mother liquor to be recycled. Measurement of the total related substances level is technically straightforward, and this value is most advantageously used to determine the optimal level of mother liquor recycle into the feed stream.

The foregoing disclosure describes the recycle of mother liquor in the first stage of recrystallization. As disclosed in pending U.S. application Serial No. 10/092,715, three or more stages of sequential recrystallization may be used to achieve an increasingly pure sucralose composition. It is recognized that the recycle process described above may also be used advantageously in earlier or later crystallization stages.

### EXAMPLES

### Example 1

Sucralose was synthesized from sucrose by the procedure disclosed in U.S. Pat. Nos. 5,470,969, 5,498,709, 4,977,294, 4,980,463, and 5,136,031, which are incorporated herein by reference. However, any method to synthesize sucralose can be used, such as those described in U.S. Pat. Nos. 4,950,746, 5,023,329, 5,298,611, and 5,530,106, which are incorporated herein by reference.

### Example 2

A sucralose solution containing related substances can be obtained by a number of previously disclosed processes for synthesizing sucralose. See, e.g., U.S. Pat. No. 5,470,969, 5,498,709, 4,977,294, 4,980,463, and 5,136,031, which are incorporated herein by reference. However, any method to synthesize sucralose can be used, such as those described in U.S. Pat. Nos. 4,950,746, 5,023,329, 5,298,611, and 5,530,106, which are incorporated herein by reference

In an embodiment of the present invention, a 6-O-acyl sucralose derivative was deacylated and steam stripped to remove any dimethylformamide remaining from the chlorination reaction. This resulted in an aqueous solution containing sucralose, residual acylated sucralose, other halogenated sugar compounds, and organic and inorganic salts.

This aqueous solution was introduced into a vessel suitable for liquid-liquid extraction, specifically, a 1 inch diameter by 12 foot tall Karr reciprocating plate column (Koch, Inc., Kansas City, Mo.). The backwash was accomplished in a 2 inch diameter by 12 foot Karr column. There were 2 foot×6 inch diameter disengaging sections at the top and bottom of the columns.

The columns were glass tubes with stainless steel plates with ⅜ inch holes supported from a central shaft. For the extraction, starting at the bottom of the column, the plates were spaced as follows: 1 foot at 6 inch spacing; 2 foot at 4 inch spacing; 1 foot at 3 inch spacing; 8 foot at 2 inch spacing. For the backwash, starting at the bottom of the column, the plates were spaced as follows: 2 foot at 4 inch spacing; 2 foot at 3 inch spacing; 7 foot at 2 inch spacing; 1 foot at 1 inch spacing.

Agitation was provided by lifting and lowering the plate stack using a connecting rod driven by an eccentric.

Positive displacement pumps were used to feed the extraction column and to empty the column bottoms. Water was supplied to the backwash column using a control valve and flow meter. The column overheads were allowed to overflow to intermediate tanks. Control was effected by varying the flow rates to maintain the interfaces in the middle of the column bottom disengaging sections. The upper interface was not controlled, but was checked from time to time for entrainment.

The aqueous solution was extracted with a volume of ethyl acetate in a ratio of 1:3.5 (ethyl acetate:aqueous solution). Although a portion of the sucralose was transferred into the ethyl acetate phase during this extraction, a proportionately greater amount of less polar impurities was removed with the ethyl acetate. Thus the majority of the sucralose remains in the aqueous solution. The ethyl acetate phase recovered from this first extraction was backwashed with water in a separate extraction step. This extraction removed a substantial portion of the sucralose, but not the related substances, from the ethyl acetate into the aqueous phase. The aqueous solution so obtained was then combined with the original feed and fed to the first extraction.

The aqueous product stream from the extraction was then extracted with fresh ethyl acetate in a suitable vessel for liquid-liquid extraction, with the ratio of ethyl acetate to water in the range of 3:1 to 4:1. One suitable vessel for this extraction is a Karr reciprocating extraction column. This extraction served to transfer the majority of the sucralose into the ethyl acetate phase, and the more polar impurities as well as the inorganic salts remained in the aqueous phase.

The ethyl acetate solution of sucralose and remaining related substances so obtained was then distilled to remove residual water present in the solution, and then sucralose was crystallized from the solution. Crystallization was facilitated by concentrating the solution as a result of evaporating a portion of the ethyl acetate, or by chilling the solution. Removal of water prior to crystallization greatly improved the rate of crystallization and the purity of the crystalline sucralose so obtained.

### Example 3

An aqueous sucralose solution obtained by alkaline deacylation of a 6-O-acetyl precursor and subsequent neutralization was introduced into a Karr column with an internal diameter of 42 inches and an agitated height of 50 feet. Water-saturated ethyl acetate was introduced into the column at a ratio of 0.35:1 (0.35 parts of ethyl acetate to 1 part aqueous sucralose solution). Two phases were obtained from the column. The ethyl acetate phase was introduced into a Karr column with a 42 inch inside diameter and an agitated height of 58 feet. Water was also introduced into the column so that the ratio of water to the ethyl acetate phase was 0.7:1.0. The aqueous solution obtained from this backwash was combined with the original feed and fed to the first extraction. The aqueous phase from the first ethyl acetate extraction was introduced into a Scheibel column (Koch, Inc., Kansas City, Mo.) with an inside diameter of 56 inches, and agitated height of 33 feet. The column contained two 24 stage separation sections. Ethyl acetate was also introduced into the column in a ratio of 3:1 (ethyl acetate:water). The ethyl acetate phase was removed, and sucralose was recovered from the ethyl acetate phase by crystallization.

What is claimed is:

1. A method for recovering sucralose from a solution comprising at least sucralose and related substances, the process comprising the following sequential steps:
(a) providing a virgin solution comprising at least sucralose and related substances;
(b) inducing the formation of sucralose crystals from the virgin solution to yield a slurry;
(c) removing a portion of the slurry;
(d) separating crystalline sucralose and a mother liquor from the portion of the slurry;
(e) recycling a portion of the mother liquor into the remaining slurry along with an additional portion of virgin solution to produce an admixture solution, wherein the portion of the recycled mother liquor is a positive number less than or equal to 0.9933−0.0321x−0.0126x wherein x is a number between 0 and 100 and is obtained by taking a value provided by dividing the weight percent of related substances in the virgin solution by the sum of the weight percent of sucralose and related substances in the virgin solution and multiplying the value by 100; and
(f) repeating steps (b)-(d) at least once.

2. A method of claim 1, wherein the inducing step (b) comprises a method selected from the group consisting of evaporative removal of water, cooling, membrane concentration, and combinations thereof.

3. A method of claim 1, wherein said crystal slurry is kept in a suspended state by continuous agitation and pumping.

4. A method of claim 1, wherein said mother liquor is substantially free of crystalline sucralose.

5. A method of claim 1, wherein the related substances is selected from the group consisting of 4,6,6′-trichlorosucrose, 4,1′,6′-trichlorosucrose, 6,1′,6′-trichlorosucrose, 4,6′-dichlorogalactosucrose, 1′,6′-dichlorosucrose, 3′,6′-dichloroanhydrosucrose, and mixtures thereof.

6. The method of claim 1, wherein said method is performed as a process selected from the group consisting of a continuous process, a semi-continuous process, and a batch process.

7. The method of claim 1, wherein the volume of virgin solution provided in step (a) is about equal to volume the portion of crystal slurry removed in step (c).

8. A method for improving the yield of crystalline sucralose in a process for recovering crystalline sucralose from a solution comprising at least sucralose and related substances, the process comprising the following sequential steps:
(a) providing a virgin solution comprising at least sucralose and related substances;
(b) inducing the formation of sucralose crystals from the virgin solution to yield a slurry;
(c) removing a portion of the slurry;
(d) separating crystalline sucralose and a mother liquor from the portion of the slurry;
(e) recycling a portion of the mother liquor into the remaining slurry along with an additional portion of virgin solution to produce an admixture solution, wherein the total related substances level relative to sucralose plus related substances in the slurry is maintained from about 6% to about 9% on a weight basis; and
(f) repeating steps (b)-(d) at least once.

9. A method of claim 8, wherein the inducing step (b) comprises a method selected from the group consisting of evaporative removal of water, cooling, membrane concentration, and combinations thereof.

10. A method of claim 8, wherein the crystal slurry is kept in a suspended state by continuous agitation and pumping.

11. The method of claim 8, wherein the mother liquor is substantially free of crystalline sucralose.

12. The method of claim 11, wherein said threshold related substances level is about 7% per weight of the virgin solution content.

13. The method of claim 8, wherein said related substances comprises a compound selected from the group consisting of 4,6,6′-trichlorosucrose, 4,1′,6′-trichlorosucrose, 6,1′,6′-trichlorosucrose, 4,6′-dichlorogalactosucrose, 1′,6′-dichlorosucrose, 3′,6′-dichloroanhydrosucrose and mixtures thereof.

14. The method of claim 8, wherein said method is performed by a process selected from the group consisting of as a continuous process, a semi-continuous process, and a batch process.

15. The method of claim 8, wherein the volume of virgin solution provided in step (a) is about equal to volume the portion of crystal slurry removed in step (c).

16. A method for enhancing the yield of crystalline sucralose from an admixture solution comprising at least sucralose, related substances, and a recycled mother liquor in a continuous process operation comprising the following sequential steps:
(a) determining the related substances level in an admixture solution comprising at least sucralose, related substances, and recycled mother liquor;
(b) providing the admixture solution into a continuous process crystallizer vessel;
(c) inducing the formation of sucralose crystals in the admixture solution to yield a slurry comprising at least sucralose crystals, sucralose, and related substances;
(d) removing a portion of the slurry;
(e) separating crystalline sucralose and a mother liquor from the portion of the slurry;
(f) determining the related substances level of the mother liquor; and
(g) recycling a portion of the mother liquor to the continuous process crystallizer vessel such that the total level of related substances level in the crystallizer vessel relative to sucralose plus related substances is about 6% to about 9% on a weight basis.

17. A method of claim 16, wherein the inducing step (b) comprises a method selected from the group consisting of evaporative removal of water, cooling, membrane concentration, and combinations thereof.

18. A method of claim 16, wherein the crystal slurry is kept in a suspended state by continuous agitation and pumping.

19. The method of claim 16, wherein said threshold related substances level is about 7% per weight of the virgin solution content.

20. The method of claim 16, wherein said related substances comprises a compound selected from the group consisting of 4,6,6′-trichlorosucrose, 4,1′,6′-trichloro sucrose, 6,1′,6′-trichlorosucrose, 4,6′-dichlorogalactosucrose, 1′,6′-dichlorosucrose, 3′,6′-dichloroanhydrosucrose and mixtures thereof.

21. The method of claim 16, wherein said method is a continuous process or a semi-continuous process.

22. The method of claim 16, wherein the volume of virgin solution provided in step (a) is about equal to volume the portion of crystal slurry removed in step (c).

23. The method of claim 16, wherein the yield of sucralose is about 50% to about 70% per weight of the total virgin solution.

24. The method of claim 23, wherein the yield of sucralose is about 55% to about 65% per weight of the total virgin solution.

25. The method of claim 24, wherein the yield of sucralose is about 50% per weight of the total virgin solution.

26. A method for enhancing the yield of crystalline sucralose from an admixture solution comprising at least sucralose, related substances, and a recycled mother liquor in a continuous process operation comprising the following sequential steps:
(a) determining the related substances level in an admixture solution comprising at least sucralose, related substances, and recycled mother liquor;
(b) providing the admixture solution into a continuous process crystallizer vessel;
(c) inducing the formation of sucralose crystals in the admixture solution to yield a slurry comprising at least sucralose crystals, sucralose, and related substances;
(d) removing a portion of the slurry;
(e) separating crystalline sucralose and a mother liquor from the portion of the slurry;
(f) determining the related substances level of the mother liquor; and
(g) recycling a portion of the mother liquor to the continuous process crystallizer vessel such that the portion of the recycled mother liquor is a positive number less than or equal to 0.9933−0.0321x−0.0126x², wherein x is a number between 0 and 100 and is obtained by taking a value provided by dividing the weight percent of related substances in the virgin solution by the sum of the weight percent of sucralose and related substances in the virgin solution and multiplying the value by 100.

27. A method of claim 26, wherein the inducing step (b) comprises a method selected from the group consisting of evaporative removal of water, cooling, membrane concentration, and combinations thereof.

28. A method of claim 26, wherein the crystal slurry is kept in a suspended state by continuous agitation and pumping.

29. The method of claim 26 wherein the mother liquor is substantially free of crystalline sucralose.

30. The method of claim 26, wherein said related substances comprises a compound selected from the group consisting of 4,6,6′-trichlorosucrose, 4,1′,6′-trichlorosucrose, 6,1′,6′-trichlorosucrose, 4,6′-dichlorogalactosucrose, 1′,6′-dichlorosucrose, 3′,6′-dichloroanhydrosucrose and mixtures thereof.

31. The method of claim 26, wherein said method is a continuous process or a semi-continuous process.

32. The method of claim 26, wherein the volume of virgin solution provided in step (a) is about equal to volume the portion of crystal slurry removed in step (c).

33. A purified sucralose composition obtained by the method of claim 1.

34. A purified sucralose composition obtained by the method of claim 8.

35. A purified sucralose composition obtained by the method of claim 16.

36. A purified sucralose composition obtained by the method of claim 26.

37. A composition comprising a solution free of solids comprising at least sucralose and related substances, wherein the level of related substances is above the level that suppresses crystal formation.

38. A composition of claim 37, wherein the amount of related substances relative to the amount of sucralose is at least 11.2 wt %.
